(19) United States
(12) Patent Application Publication
(10) Pub. No.: US 2024/0247007 A1
(43) Pub. Date: Jul. 25, 2024

(54) CHEMICALLY LINKABLE NUCLEAR TARGETING TAGS

(71) Applicant: SIGMA-ALDRICH CO. LLC, St. Louis, MO (US)

(72) Inventors: David Klick, Elmhurst, IL (US); Kaelyn Wilke, Wauwatosa, WI (US); Ashwini Ghogare, Somerville, MA (US); Ravindra Vikram Singh, Bangalore (IN)

(21) Appl. No.: 18/558,693

(22) PCT Filed: May 4, 2022
(86) PCT No.: PCT/US2022/072102

§ 371 Date: Nov. 2, 2023

Related U.S. Application Data
(60) Provisional application No. 63/183,894, filed on May 4, 2021.

(51) Int. Cl. C07F 5/04; A61K 47/54; C07F 5/02

(52) U.S. Cl. CPC C07F 5/04; A61K 47/54; A61K 47/545; C07F 5/025

(57) ABSTRACT

Nuclear targeting tags having a phenylboronate targeting moiety, a linker, a chemically linkable end group suitable for linking the targeting moiety to a molecule of interest, and optionally a spacer between the linker and chemically linkable end group. Also provided are compounds including the nuclear targeting tag covalently bonded to a molecule of interest, such as a drug, probe, dye, peptide, protein, drug candidate, or natural product. Also provided are methods of introducing a molecule of interest into a nucleus of a cell using the nuclear targeting tag. Also provided are methods for preparing the nuclear targeting tags.

### CROSS-REFERENCE TO RELATED APPLICATIONS

The present application claims the benefit of priority of U.S. provisional patent application No. 63/183,894, filed on May 4, 2021, the entire content of which is incorporated herein in its entirety.

Delivery of molecules of interest, such as drugs, probes, dyes, peptides, proteins, and so forth, to the nucleus of a cell is of great interest to researchers across a wide variety of disciplines. For example, targeting research probes to the nucleus will allow for further elucidation of organelle-specific molecular mechanisms. Nuclear targeting of therapeutics is desired to increase therapeutic efficacy while reducing off-target side effects.

Conventional methods involve the use of short peptides or proteins to transport the molecule of interest into the nucleus. However, several problems with the use of such peptides or proteins exist, including instability, short shelf life, incompatibility with some molecules of interest, difficulty in linking to some molecules of interest, and difficultly in manufacture.

A need exists for new nuclear targeting tags. Such nuclear targeting tags should have higher stability and shelf life, be able to easily be linked to a myriad of molecules of interest and be able to be manufactured in a cost-effective manner.

### SUMMARY

Provided are nuclear targeting tags having a phenylboronate targeting moiety, a linker, attached to the phenyl group of the phenylboronate targeting moiety, and a chemically linkable end group. The linker is selected from a carbamate, an ether, an alkylenyl group, and a PEG group. In certain embodiments, the phenylboronate targeting moiety is a phenylboronic acid group; in other embodiments, the phenylboronate targeting moiety is a phenylboronic acid pinacol ester group.

In some embodiments, the nuclear targeting tag also includes a spacer between the linker and the chemically linkable end group. Preferred spacers include alkylenyl spacers and PEG spacers.

The chemically linkable end group may be selected from halo, amino, carboxyl, C₂-C₁₀ alkynyl, hydroxyl, C₁-C₁₀ alkoxy, azido, sulfinate, thiol, fluorosulfate, and boronic acid.

In some embodiments, the nuclear targeting tag may also include a molecule covalently bound to the chemically linkable end group. Suitable molecules include, but are not limited to drugs, probes, dyes, peptides, proteins, drug candidates and natural products.

In one embodiment, a compound of Formula I is provided:

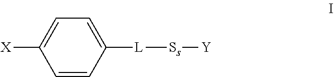

wherein X is a boronic acid or boronic ester, L is a linker, S is a spacer, s is 0 to 10, and Y is a chemically linkable end group.

In preferred embodiments, X is selected from a boronic acid group and a boronic acid pinacol ester group, L is selected from a carbamate, an ether, a C₁-C₂₀ alkylenyl group, and —[—O—CH₂CH₂-]ₙ⁻, wherein n is 1 to 12, S is selected from C₁-C₂₀ alkylenyl group and —[—O—CH₂CH₂-]ₙ⁻, wherein n is 1 to 12, s is 0 to 10, and Y is selected from halo, amino, carboxyl, C₂-C₁₀ alkynyl, hydroxyl, C₁-C₁₀ alkoxy, azido, sulfinate, thiol, fluorosulfate, and boronic acid.

In some embodiments, the compound of Formula I further including a molecule conjugated to the chemically linkable end group. Such molecules may include drugs, probes, dyes, peptides, proteins, drug candidates and natural products.

In a second embodiment, a compound of Formula II is provided

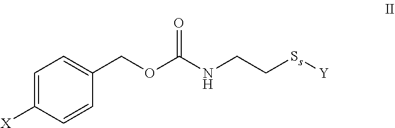

wherein X is a boronic acid or boronic ester, S is a spacer, s is 0 to 10, and Y is a chemically linkable end group. In a preferred embodiment, X is selected from a boronic acid group and a boronic acid pinacol ester group, S is selected from C₁-C₂₀ alkylenyl and —[—O—CH₂CH₂—]ₙ⁻, wherein n is 1 to 12 and s is 0 to 10, and Y is selected from halo, amino, carboxyl, C₂-C₁₀ alkynyl, hydroxyl, C₁-C₁₀ alkoxy, azido, sulfinate, thiol, fluorosulfate, and boronic acid.

In some preferred embodiments, the compound of Formula II also includes a molecule conjugated to the chemically linkable end group. In such embodiments, the molecule is selected from the group consisting of drugs, probes, dyes, peptides, proteins, drug candidates and natural products.

Also provided is a compound of Formula III

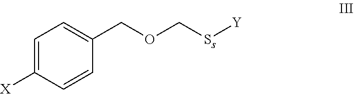

wherein X is a boronic acid or boronic ester, S is a spacer, s is 0 to 10, and Y is a chemically linkable end group. In a preferred embodiment, X is selected from a boronic acid group and a boronic acid pinacol ester group, S is selected from C₁-C₂₀ alkylenyl and —[—O—CH₂CH₂—]ₙ⁻, wherein n is 1 to 12 and s is 0 to 10, and Y is selected from halo, amino, carboxyl, C₂-C₁₀ alkynyl, hydroxyl, C₁-C₁₀ alkoxy, azido, sulfinate, thiol, fluorosulfate, and boronic acid.

In some preferred embodiments, the compound of Formula II also includes a molecule conjugated to the chemically linkable end group. In such embodiments, the molecule is selected from the group consisting of drugs, probes, dyes, peptides, proteins, drug candidates and natural products.

Further provided are methods of delivering a molecule to a nucleus in a cell by contacting the cell with a nuclear targeting tag of Formula I or II that includes the molecule of interest bound to the nuclear targeting tag.

Further provided are methods of preparing the nuclear targeting tags described herein.

### DETAILED DESCRIPTION

Provided are compounds that include a phenylboronate nuclear targeting moiety linked to a conjugation moiety that allows for conjugation of many different molecules of interest, indeed many different classes of molecules of interest, to the targeting moiety, allowing for import of the molecule of interest into a cell nucleus, where it can then provide many different functions.

In some embodiments, the phenylboronate targeting moiety may be conjugated, through a linker and optionally a spacer, to a small molecule, i.e., a drug, probe, or dye, while in other embodiments, it could also be used with peptides or proteins.

The resulting tag-bearing compound allows targeting to and enrichment in the nucleus over other cellular compartments. Targeting research probes to the nucleus may allow further elucidation of organelle-specific molecular mechanisms whereas nuclear targeting of therapeutics can increase therapeutic efficacy while reducing off-target side effects. The compounds provided herein have the benefit of higher stability and longer shelf lives than current nuclear targeting tags which are predominantly short amino acid peptides or proteins. In addition, these nuclear-targeting tags are smaller, more easily linked to one's molecule of interest, and cheaper to manufacture.

The nuclear targeting tags include at one end a phenyl boronate targeting moiety, and a chemically linkable end group at the other end, joined by a linker, and optionally, a spacer.

In certain embodiments, the phenylboronate targeting moiety is a boronic acid group; in other embodiments, the phenylboronate targeting moiety is a phenylboronic acid pinacol ester, i.e., 4,4,5,5-tetramethyl-1,3,2-diozaborolan2-yl, group. Other arylboronate targeting moieties may be envisioned by those skilled in the art.

The linker is selected from a carbamate, an ether, an alkylenyl group and a polyethylene glycol (PEG). As used herein, the term alkylenyl refers to a divalent analog of a linear or branched alkyl group. In embodiments in which the linker is an alkylenyl group, the linker may be a C₁-C₂₀ alkylenyl. In some embodiments, the linker may be a C₁-C₁₀ alkylenyl. In some embodiments, the alkylenyl linker is a straight chain alkyl, in other embodiments the alkylenyl linker may be a branched alkylenyl. In embodiment in which the linker is a PEG, it may include from 1 to 12 PEG units. In preferred embodiments, the linker is in the para-position relative to the boronate group.

In some embodiments, the nuclear targeting tag also includes a spacer between the linker and the chemically linkable end group. The spacer may be used, for example, to provide separation between the phenylboronate targeting moiety and the molecule of interest, allowing the molecule of interest to interact with its target within the nucleus. In some embodiments, the spacer may incorporate a disulfide group, a photolabile group, or other group that allows for release of the molecule of interest once it is inside the nucleus. Preferred spacers include C₁-C₂₀ alkylenyl, and —[—O—CH₂CH₂—]ₙ⁻, wherein n is 1 to 12, though other spacers may be envisioned, either as equivalent or to introduce additional functionality.

The chemically linkable end group may be selected from many different end groups that allow conjugation or chemical bond to the molecule of interest. Some preferred chemically linkable end groups include, but are not limited to halo, amino, carboxyl, C₂-C₁₀ alkynyl, hydroxyl, C₁-C₁₀ alkoxy, azido, sulfinate, thiol, fluorosulfate, and boronic acid.

In some embodiments, the nuclear targeting tag may also include a molecule covalently bound to the chemically linkable end group. Suitable molecules include, but are not limited to drugs, probes, dyes, peptides, proteins, drug candidates and natural products.

Examples of drugs and drug candidates include small molecule drugs, peptide drugs, natural products, protein therapeutics, antibodies, antibody-like bind agents, and antibody fragments, such as single chain antigen binding fragments. Specific drugs may include but are not limited to rebimastat, fluconazole, tanomastat and similar. Specific peptide drugs may include but are not limited to ziconotide, enfuvirtide, octreotide, lanreotide, and pasireotide. Drug candidates may include any small molecules or peptides that are selected for or are going through clinical trials.

Peptides suitable for use with the nuclear targeting tags described herein may include any peptides having 2 or more amino acids, substituted amino acids, amino acid derivatives, or substituted amino acid derivatives, including standard, non-standard, and chemically synthesized amino acids, and including for L- and D-isomers, that are linked together in an amide linkage. Substituted amino acids are amino acids that include one or more substituents, typically on the side chain. Amino acid derivatives are amino acids which the α-amino group or acyl group have been chemically modified. An exemplary amino acid for use with the nuclear targeting tags described herein is azatyrosine.

Proteins suitable for use with the nuclear targeting tags described herein include natural proteins, including but not limited to antibodies, synthetic proteins, and protein fragments. Some exemplary proteins include but are not limited to gemtuzumab and brentuximab.

Examples of suitable probes for use with the nuclear targeting tags described herein include but are not to single-stranded DNA or RNA that binds a complementary DNA sequence, histones, nuclear transport proteins and nuclear location proteins.

Examples of suitable dyes for use with the nuclear targeting tags described herein include but are not limited to heptamethine, cyanine and fluoresceine.

Examples of suitable natural products for use with the nuclear targeting tags described herein include but are not limited to anthramycins and maytansinoids.

In one embodiment, a compound of Formula I is provided:

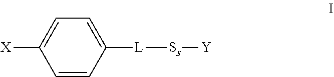

wherein X is a boronic acid or boronic ester, L is a linker, Sₛ is a spacer and Y is a chemically linkable end group.

In preferred embodiments, X is selected from a boronic acid group and a boronic acid pinacol ester, i.e., 4,4,5,5-tetramethyl-1,3,2-diozaborolan2-yl, group.

Linker L may be a carbamate group, an ether, a C₁-C₂₀ alkylenyl group, or —[—O—CH₂CH₂—]ₙ⁻, wherein n is 1 to 12. In one preferred embodiment, linker L is a carbamate group. In another preferred embodiment, linker L is an ether.

Spacer Sₛ is an optional spacer unit. Use of spacer Sₛ and choice of specific spacer will depend on the molecule of interest, its function, and preserving the interaction of the linked molecule to its target. The spacer unit provides added tunability to the nuclear localization tags provided herein and may be varied to provide optimal structure for localization depending on both the molecule and the nuclear target.

In some embodiments when spacer Sₛ is present, spacer Sₛ is selected from C₁-C₂₀ alkylenyl group, preferably C₄-C₁₈ alkylenyl group; and s is 1 to 10. When spacer Sₛ is alkylenyl, it may be straight chain or branched. In some embodiments when spacer Sₛ is alkylenyl, it may include one or more substituents. In some embodiments, when spacer Sₛ is alkylenyl, it may include one or more unsaturated bonds. In other embodiments when spacer Sₛ is present, spacer Sₛ is a PEG of formula —[—O—CH₂CH₂—]ₙ⁻, wherein n is 1 to 12, preferably 4 to 12, and s may be 1 to 10. In still other embodiments, the spacer Sₛ may be made of multiple alkylenyl units, PEG units or combination of alkylenyl units and PEG units. In such embodiments, s may be 2 to 10.

The chemically linkable end group Y is selected to provide a convenient way to conjugate to or form a covalent bond with a molecule of interest. In various embodiments, the chemically linkable end group may be halo, e.g., fluoro, chloro, bromo, iodo; amino, including primary amines, secondary amines, and amino acids; carboxyl, including organic acids and amino acids; C₂-C₁₀ alkynyl, such as methynyl, ethynyl, propynyl, butynyl and so forth; hydroxyl; C₁-C₁₀ alkoxy, including, for example, methoxy, ethoxy, propoxy, butoxy, and so forth; azido, sulfinate, thio, fluorosulfate, and boronic acid.

In some embodiments, the compound of Formula I further includes a molecule conjugated to the chemically linkable end group. Such molecules may include drugs, probes, dyes, peptides, proteins, drug candidates and natural products, each as outlined above.

In a second embodiment, a compound of Formula II is provided

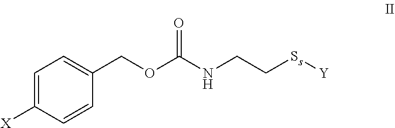

wherein X is a boronic acid or boronic ester, S is an optional spacer, with s from 0 to 10, and Y is a chemically linkable end group. In a preferred embodiment, X is selected from a boronic acid group and a boronic acid pinacol ester, i.e., 4,4,5,5-tetramethyl-1,3,2-diozaborolan2-yl, group.

When spacer S is present, it is preferably selected from an alkylenyl spacer and a PEG spacer. When S is an alkylenyl spacer, it is preferably a C₁-C₂₀ alkylenyl group. When S is a PEG spacer of formula —[—O—CH₂CH₂—]ₙ, n is preferably 1 to 12. In some embodiments, the spacer S may be made of multiple alkylenyl units, PEG units or combination of alkylenyl units and PEG units. In such embodiments, s may be 2 to 10.

The chemically linkable end group Y is selected to provide a convenient way to conjugate to or form a covalent bond with a molecule of interest as described above. In various embodiments, the chemically linkable end group may be halo, e.g., fluoro, chloro, bromo, iodo; amino, including primary amines, secondary amines, and amino acids; carboxyl, including organic acids and amino acids; C₂-C₁₀ alkynyl, such as methynyl, ethynyl, propynyl, butynyl and so forth; hydroxyl; C₁-C₁₀ alkoxy, including, for example, methoxy, ethoxy, propoxy, butoxy, and so forth; azido, sulfinate, thio, fluorosulfate, and boronic acid.

In some preferred embodiments, the compound of Formula II also includes a molecule conjugated to the chemically linkable end group. In such embodiments, the molecule is selected from the group consisting of drugs, probes, dyes, peptides, proteins, drug candidates and natural products.

In some preferred embodiments, the nuclear targeting tags provided herein may be (4-((((2-Aminoethyl)carbamoyl)oxy)methyl)phenyl)boronic acid hydrochloride, (4-((((2-Bromoethyl)carbamoyl)oxy)methyl)phenyl)boronic acid, 4-(4,4,5,5-Tetramethyl-1,3,2-dioxaborolan-2-yl)benzyl (bromoethyl)carbamate, 4-(4,4,5,5-Tetramethyl-1,3,2-dioxaborolan-2-yl)benzyl (2-aminoethyl)carbamate and 2-((4-(4,4,5,5-Tetramethyl-1,3,2-dioxaborolan-2-yl)benzyl)oxy)ethan-1-amine.

Further provided are methods of delivering a molecule to a nucleus in a cell by contacting the cell with a nuclear targeting tag of Formula I or II that includes the molecule of interest bound to the nuclear targeting tag.

Also included are methods of delivering a molecule of interest to a cell nucleus in a subject, the method including the step of administering a nuclear targeting tag as described herein, including the molecule of interest bound to the chemically linkable end group, to the subject in need of such treatment. In various embodiment, the subject may be a human subject or a veterinary subject.

The present disclosure further provides the following embodiments set forth in the clauses below.

{1} A first method for preparing a nuclear targeting tag, the method comprising the steps of
a. reacting a pinocol boronate phenyl alcohol with a carbonyldiimidazole to form a boronate carbonyldiimidazole intermediate, and
b. reacting the boronate carbonyldiimidazole intermediate of step (a) with ethylene diamine to form the nuclear targeting tag.

{2} The method of clause {1} wherein step (a) is carried out in acetonitrile.

{3} The method of either of clauses {1} or {2} wherein step (a) is carried out at an elevated temperature, preferably 50° C.

{4} The method of any of clauses {1} to {3} wherein step (b) is carried out in tetrahydrofuran (THF).

{5} The method of any of clauses {1} to {4} further comprising the step of
c. purifying the nuclear targeting tag.

{6} A method for preparing a nuclear targeting tag, the method comprising the steps of
a. reacting pinocol boronate phenyl alcohol (1) with a carbonyldiimidazole (2) to form a boronate carbonyldiimidazole intermediate (3)

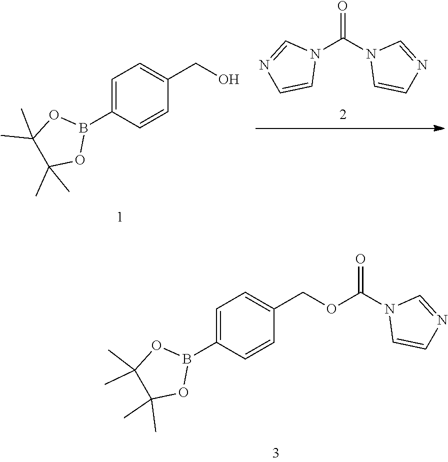

and
b. reacting the boronate carbonyldiimidazole intermediate (3) with ethylene diamine to form the nuclear targeting tag (4)

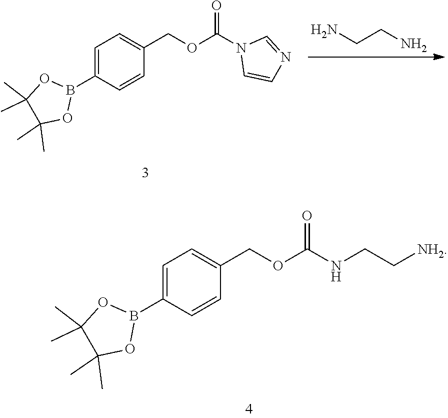

{7} A second method for preparing a nuclear targeting tag, the method comprising the steps of
a. reacting pinacol boronate phenyl alcohol with a carbonyldiimidazole to form a boronate carbonyldiimidazole intermediate, and
b. reacting the boronate carbonyldiimidazole intermediate of step (a) with a bromoalkylamine hydrobromide to form the nuclear targeting tag.

{8} The method of clause {7} wherein step (a) is carried out in acetonitrile.

{9} The method of either of clauses {7} or {8} wherein step (a) is carried out at an elevated temperature, preferably 50° C.

{10} The method of any of clauses {7} to {9} wherein step (b) is carried out in dimethylformamide (DMF).

{11} The method of any of clauses {7} to {10} further comprising the step of
c. purifying the nuclear targeting tag.

{12} A method for preparing a nuclear targeting tag, the method comprising the steps of
a. reacting pinacol boronate phenyl alcohol (1) with a carbonyldiimidazole (2) to form boronate carbonyldiimidazole intermediate (3)

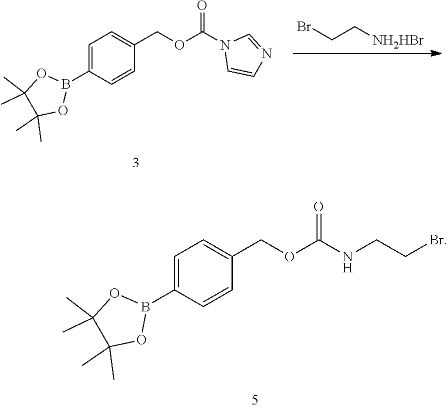

and
b. reacting the boronate carbonyldiimidazole intermediate (3) with ethylene diamine to form the nuclear targeting tag (5)

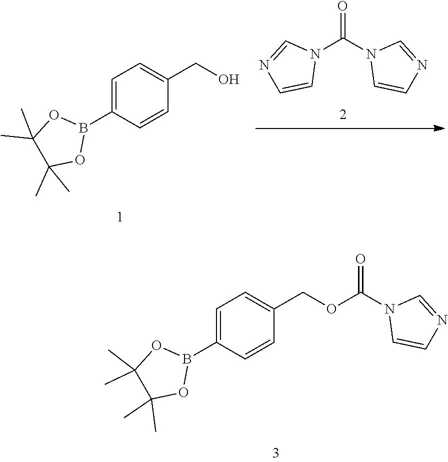

{13} A third method for preparing a nucleus tag, the method comprising the steps of
a. reacting a pinacol boronate phenyl alcohol with a carbonyldiimidazole to form a boronate carbonyldiimidazole intermediate,
b. reacting the boronate carbonyldiimidazole intermediate of step (a) with an amine-protected ethylene diamine to form an amine-protected-pinacolyl boronate ester intermediate,
c. reacting the amine-protected-pinacolyl boronate ester intermediate of step (b) with diethanolamine to form an amine-protected-pinacolyl borate DEA complex,
d. reacting the amine-protected-pinacolyl borate DEA complex of step (c) with diethylether to form the nuclear targeting tag, and
e. optionally deprotecting the amine.

{14} The method of clause {13} wherein step (a) is carried out in acetonitrile.

{15} The method of either of clauses {13} or {14} wherein step (a) is carried out at an elevated temperature, preferably 50° C.

{16} The method of any of clauses {13} to {15} wherein step (b) is carried out in THF.

{17} The method of any of clauses {13} to {16} wherein step (c) is carried out in presence of isopropyl alcohol.

{18} The method of any of clauses {13} to {17} wherein step (c) is carried out in diethyl ether.

{19} The method of any of clauses {13} to {18} wherein step (d) is carried out in the presence of isopropyl alcohol.

{20} The method of any of clauses {13} to {19} wherein step (d) is carried out in acetonitrile.

{21} The method of any of clauses {13} to {20} further comprising the step of purifying the nuclear targeting tag.

{22} A method for preparing a nucleus tag, the method comprising the steps of
a. reacting a pinacol boronate phenyl alcohol (1) with a carbonyldiimidazole (2) to form a boronate carbonyldiimidazole intermediate (3).

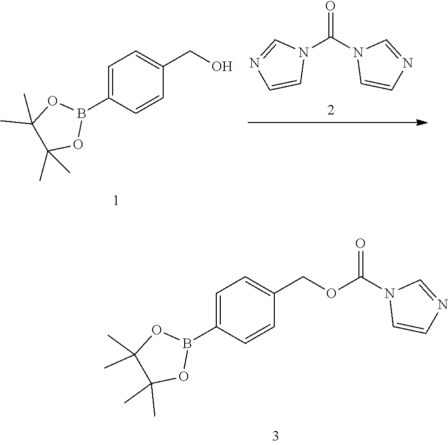

b. reacting the boronate carbonyldiimidazole intermediate (3) with a protected ethylene diamine to form an amine-protected-pinacolyl diethanolamine intermediate (6),

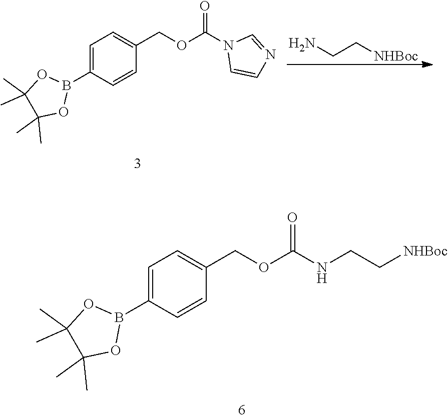 [SMILES: CC1(C)OB(c2ccc(COC(=O)n3ccnc3)cc2)OC1(C)C ; CC(C)(C)OC(=O)NCCN ; CC(C)(C)OC(=O)NCCNC(=O)OCc1ccc(B2OC(C)(C)C(C)(C)O2)cc1]

c. reacting the amine-protected-pinacolyl boronate ester intermediate (6) with diethanolamine to form an amine-protected-pinacolyl borate DEA complex (7),

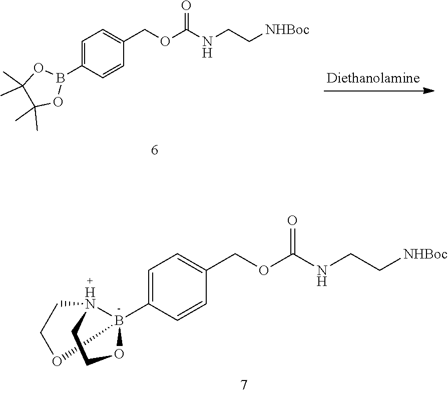

and
d. reacting the amine-protected-pinacolyl borate DEA complex (7) with diethylether and deprotecting the amine to form the nuclear targeting tag (8)

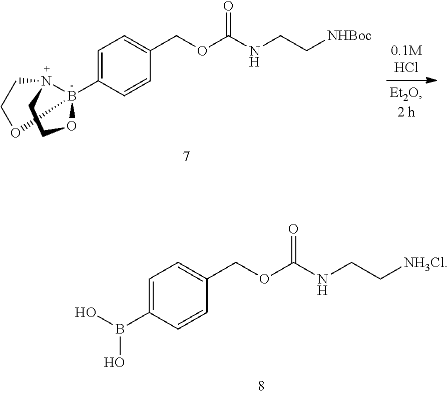

{23} A fourth method of preparing a nuclear targeting tag, the method comprising the steps of
a. reacting a pinacol boronate phenyl alcohol with a carbonyldiimidazole to form a boronate carbonyldiimidazole intermediate,
b. reacting the boronate carbonyldiimidazole intermediate of step (a) with a bromoalkylamine hydrobromide to form a bromo-pinacolyl boronate ester intermediate,
c. reacting the bromo-pinacolyl boronate ester intermediate of step (b) with diethanolamine to form a bromo-pinacolyl borate DEA complex,
d. reacting the bromo-pinacolyl borate DEA complex of step (c) with diethylether to form the nuclear targeting tag.

{24} The method of clause {23} wherein step (a) is carried out in acetonitrile.

{25} The method of either of clauses {23} or {24} wherein step (a) is carried out at an elevated temperature, preferably 50° C.

{26} The method of any of clauses {23} to {25} wherein step (b) is carried out in DMF.

{27} The method of any of clauses {23} to {26} wherein step (b) is carried out in the presence of diethyl ether.

{28} The method of any of clauses {23} to {27} wherein step (c) is carried out in the presence of isopropyl alcohol.

{29} The method of any of clauses {23} to {28} wherein step (c) is carried out in diethyl ether.

{30} The method of any of clauses {23} to {29} wherein step (d) is carried out in diethyl ether.

{31} The method of any of clauses {23} to {30} wherein step (d) is carried out in the presence of an acid, preferably hydrochloric acid.

{32} The method of any of clauses {23} to {31} further comprising the step of
e. purifying the nuclear targeting tag.

{33} A method of preparing a nuclear targeting tag, the method comprising the steps of
a. reacting a pinacol boronate phenyl alcohol (1) with a carbonyldiimidazole (2) to form a boronate carbonyldiimidazole intermediate (3),

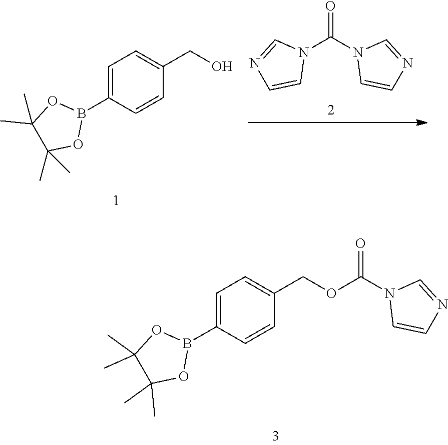

b. reacting the boronate carbonyldiimidazole intermediate (3) with a bromoalkylamine hydrobromide to form a bromo-pinacolyl boronate ester intermediate (9),

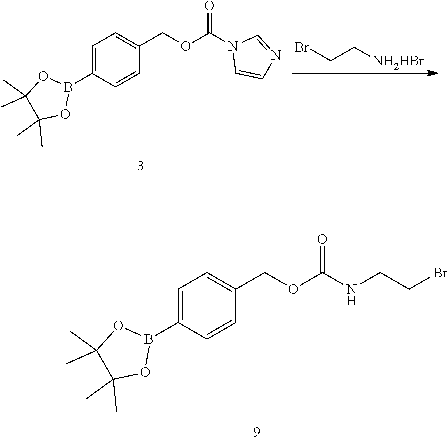

c. reacting the bromo-pinacolyl boronate ester intermediate (9) with diethanolamine to form a bromo-pinacolyl borate DEA complex (10),

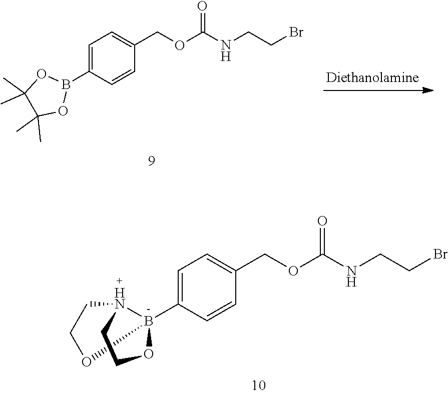

d. reacting the bromo-pinacolyl borate DEA complex (10) with diethylether to form the nuclear targeting tag (11)

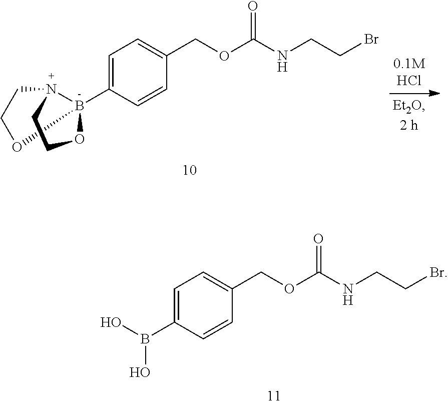

{34} A fifth method of preparing a nuclear targeting tag, the method comprising the steps of
a. reacting N-(2-hydroxyethyl)phthalimide with 4-bromobenzyl bromide with to form a 2-(2(4-bromobenzyl)oxyethyl isoindoline-1,3-dione intermediate,
b. reacting the 2-(2(4-bromobenzyl)oxyethyl isoindoline-1,3-dione intermediate of step (a) with bis-pinacol boronate to form a 2-(2-(4,4,5,5-tetramethyl-1,3,2-dioxoboronate-2-yl)benzyl)oxy)ethyl)isoindoline-1,3-dione intermediate,
c. reacting the 2-(2-(4,4,5,5-tetramethyl-1,3,2-dioxoboronate-2-yl)benzyl)oxy)ethyl)isoindoline-1,3-dione intermediate of step (c) with hydrazine hydrate to form the nuclear targeting tag.

{35} The method of clause {34} wherein step (a) is carried out in DMF.

{36} The method of either of clauses {34} or {35} wherein step (a) is carried out in the presence of sodium hydride.

{37} The method of clause {36} wherein the DMF and sodium hydride are combined and cooled, preferably to 0° to 5° C. prior to adding the N-(2-Hydroxyethyl)phthalimide.

{38} The method of clause {37} wherein the N-(2-Hydroxyethyl)phthalimide in DMF and NaH is heated, preferably to 40° C., prior to addition of 4-bromo benzyl bromide.

{39} The method of clause {38} wherein the reaction of step (a) is carried out at an elevated temperature, preferably 70° C.

{40} The method of any of clauses {34} to {39} wherein step b is carried out in the presence of a catalyst.

{41} The method of clause {40} wherein the catalyst is a palladium catalyst.

{42} The method of clause {41} wherein the catalyst is PdCl₂(dppf).

{43} The method of any of clauses {34} to {42} wherein step (b) is carried out in dichloromethane.

{44} The method of any of clauses {34} to {43} wherein step (b) is carried out in the presence of potassium acetate.

{45} The method of any of clauses {34} to {44} wherein step (b) is carried out at an elevated temperature, preferably 85° C.

{46} The method of any of clauses {34} to {45} wherein step (c) is carried out in ethanol,

{47} The method of any of clauses {34} to {46} further comprising the step of
d. purifying the nuclear targeting tag.

{48} A method of preparing a nuclear targeting tag, the method comprising the steps of
a. reacting 4-bromobenzyl bromide (12) with N-(2-hydroxyethyl)phthalimide (13) to form a 2-(2(4-bromobenzyl)oxyethyl isoindoline-1,3-dione intermediate (14),

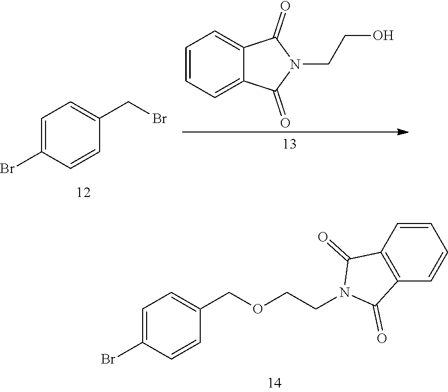 [SMILES: BrCc1ccc(Br)cc1 ; O=C1c2ccccc2C(=O)N1CCO ; O=C1c2ccccc2C(=O)N1CCOCc1ccc(Br)cc1]

b. reacting the 2-(2(4-bromobenzyl)oxyethyl isoindoline-1,3-dione intermediate (14) with bis-pinacol boronate, in the presence of a catalyst, to form a 2-(2-(4,4,5,5-tetramethyl-1,3,2-dioxoboronate-2-yl)benzyl)oxy)ethyl)isoindoline-1,3-dione intermediate (15),

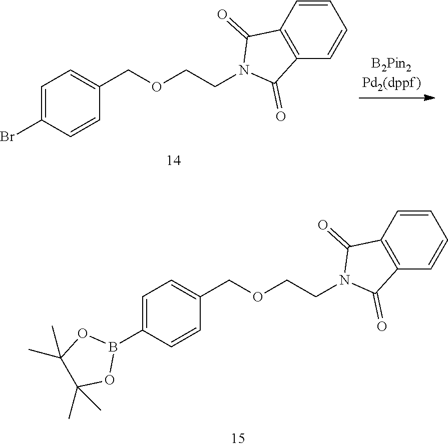 [SMILES: O=C1c2ccccc2C(=O)N1CCOCc1ccc(Br)cc1 ; CC1(C)OB(c2ccc(COCCN3C(=O)c4ccccc4C3=O)cc2)OC1(C)C]

c. reacting the 2-(2-(4,4,5,5-tetramethyl-1,3,2-dioxoboronate-2-yl)benzyl)oxy)ethyl)isoindoline-1,3-dione intermediate (15) with hydrazine hydrate to form the nuclear targeting tag (16).

### EXAMPLES

### Example 1. Synthesis of 4-(4,4,5,5-tetramethyl-1,3,2-dioxaborolan-2-yl)benzyl (2-aminoethyl)carbamate

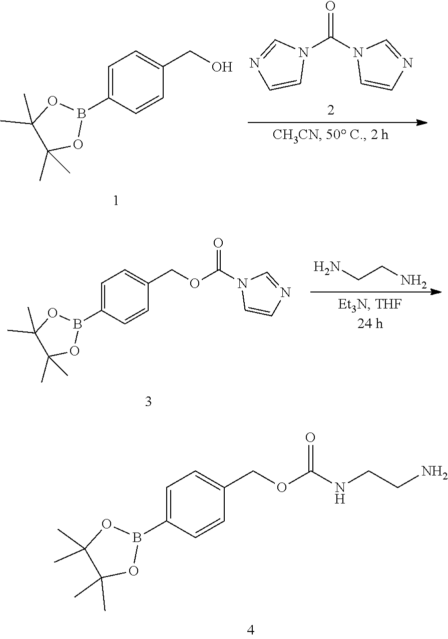

A 500 mL 4-neck round bottom flask was flame dried and equipped with condenser, thermo-pocket, septum and magnetic stir bar. The whole system was evacuated with nitrogen gas. Boronate alcohol (20 g, 85 mmol) carbonyldiimidazole (18 g, 111 mmol) and 200 ml acetonitrile were charged to the flask; the reaction mass was stirred for approximately 2 hours at 50° C. After completion of the reaction as indicated by TLC, the reaction mass was cooled, and acetonitrile distilled out under reduced pressure at 40° C. 50 mL n-hexane was charged to the reaction vessel and stirred for 30 minutes. The reaction mass was filtered to get a white colored solid. The material was dried under vacuum for 2 h at 40° C. to obtain 26 g white product.

A 500 mL 4 neck round bottom flask was flame dried and equipped with nitrogen bubbler, septum and thermometer. Boronate CDI (10 g, 30 mmol), in 250 ml THF 20 ml was charged to the flask. Ethylene diamine (20 ml, 300 mmol) was added to the flask and reaction mixture was stirred at room temperature for 24 hours. After completion of the reaction as indicated by TLC, the solvent was evaporated, 25 mL dichloromethane and 10 ml water added to residue. The reaction was stirred for 5 mins. The mixture separated into two layers. The organic layer washed with water and dried over sodium sulphate. The organic layer was concentrated under reduced pressure to yield 3 g of the title compound.

### Example 2. Synthesis of 4-(4,4,5,5-tetramethyl-1,3,2-dioxaborolan-2-yl)benzyl (2-bromoethyl)carbamate

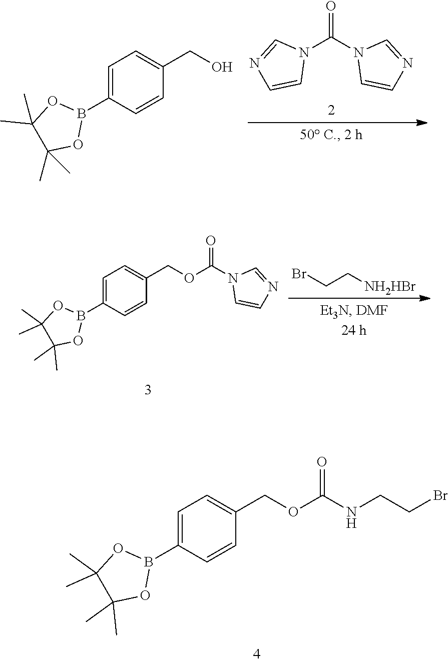

A 1 L three neck round bottom flask fitted with reflux condenser, nitrogen bubbler, and thermometer. Carbonyldiimidazole (18 g, 111 mmoles) in dry acetonitrile (200 mL) was added to the flask. Pinacol boronate phenyl alcohol (20 g, 85 mmol) was added to the reaction flask under nitrogen atmosphere. The resulting mixture was heated to 50° C. and allowed to react until TLC analysis indicated no starting material was present, approximately 2 hours. After the completion of reaction, the reaction mixture was cooled to RT and concentrated under reduced pressure to afford yellowish residue. The crude residue was triturated using pentane or hexane to afford 25 g of carbamate as a white solid.

A 500 mL 4 neck round bottom flask was flame dried and equipped with condenser, thermopocket, septum and magnetic stir bar. The system was evacuated with N₂ gas for 15 minutes. Boronate CDI (12 g, 36.5 mmol), 2-bromoethylamine hydrobromide (19 g, 92 mmol) and 120 mL (10.0 vol) dry DMF were charged to the reaction flask. The reaction mass was stirred for 10 minutes to yield a homogeneous clear solution, then triethylamine (15 g, 146 mmol) was charged to the reaction flask by syringe. The resulting heterogeneous mixture was stirred until the reaction was complete, as indicated by TLC. After completion, 360 mL diethyl ether charged to the reaction mass and stirred for 10 mins. The reaction mass was filtered and the filtrate was extracted with ice water, brine and dried over anhydrous sodium sulphate. The organic layer concentrated under reduced pressure to yield crude product, which was purified by silica gel chromatography to yield 5.3 g of the solid title product.

### Example 3. Synthesis of 4-(4,4,5,5-tetramethyl-1,3,2-dioxaborolan-2-yl)benzyl (2-bromoethyl)carbamate hydrochloride

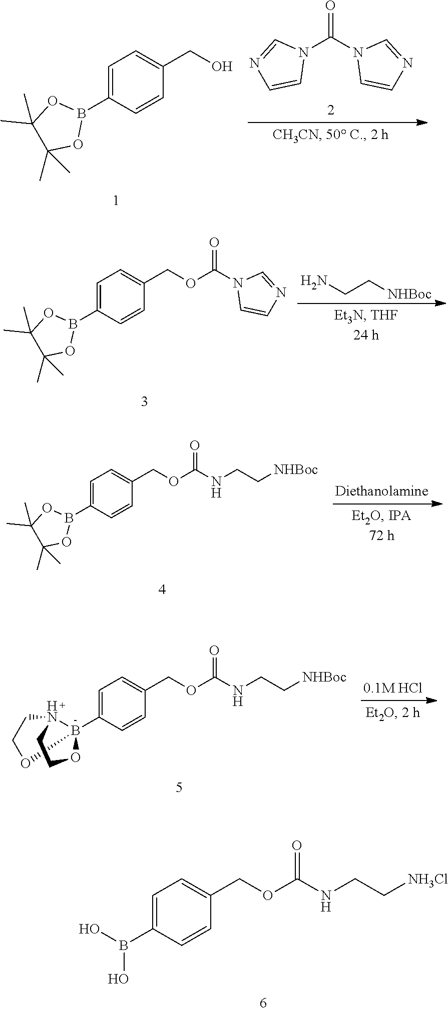

A 1 L three neck round bottom flask was fitted with reflux condenser, nitrogen bubbler, and thermometer, and carbonyldiimidazole (18 g, 111 mmoles) was in dry acetonitrile (200 mL) was added. Pinacol boronate phenyl alcohol (20 g, 85 mmol) was added to the reaction mixture under nitrogen atmosphere. The resulting mixture was heated to 50° C. and reacted until TLC analysis indicated no starting material, approximately 2 hours. After the completion of reaction, the reaction mixture was cooled to RT and concentrated under reduced pressure to afford yellowish residue. The crude residue was triturated using pentane or hexane to afford 25 g of carbamate as a white solid.

To a solution of imidazole carbamate (20 g, 61 mmol) in 300 mL dry THF, in a 1 L three neck round bottom flask fitted with nitrogen bubbler, thermometer and stopper, N-Boc-ethylene diamine (18 g, 74 mmol) was added. The reaction mixture was stirred under nitrogen at room temperature for approximately 20 hours. The solvent was removed under reduced pressure and the residue was purified by silica gel column chromatography to provide 18 g product as a colorless low melting solid.

A 1 L three neck round bottom flask was flame dried, fitted with nitrogen bubbler, thermometer and septum, amd evacuated with nitrogen gas for 15 minutes. N-boc pinacolyl boronate ester (16 g, 38 mmol), diethanol amine (5 g, 45 mmol), 32 ml IPA and 480 ml diethyl ether were charged to the flask under nitrogen atmosphere. The resulting mixture was stirred at room temperature for approximately 72 hours. After the completion of reaction, the reaction mixture was filtered, and the product washed with diethyl ether. Finally, the product was dried under vacuum at 45 degrees to obtain 14 g white solid.

A 250 ml three neck round bottom flask was flame dried, fitted with nitrogen bubbler, thermometer and septum, and was evacuated with nitrogen gas for 15 mins. To this flask was charged N-boc-pinacolyl boronate DEA complex (6.0 g, 15 mmol), 180 mL of 0.1 M diethyl ether, 12 mL IPA and 300 mL acetonitrile. The reaction was stirred at room temperature for 2 hours and kept overnight at room temperature. After the completion of reaction, the organics were distilled and the residue dissolved in 300 mL diethyl ether. The ether layer was washed with water, dried over Na₂SO₄ and concentrated. 3.5 g of the title compound was obtained as white crystalline material.

### Example 4. Synthesis of (4-((((2-bromoethyl)carbamoyl)oxy)methyl)phenyl)boronic acid

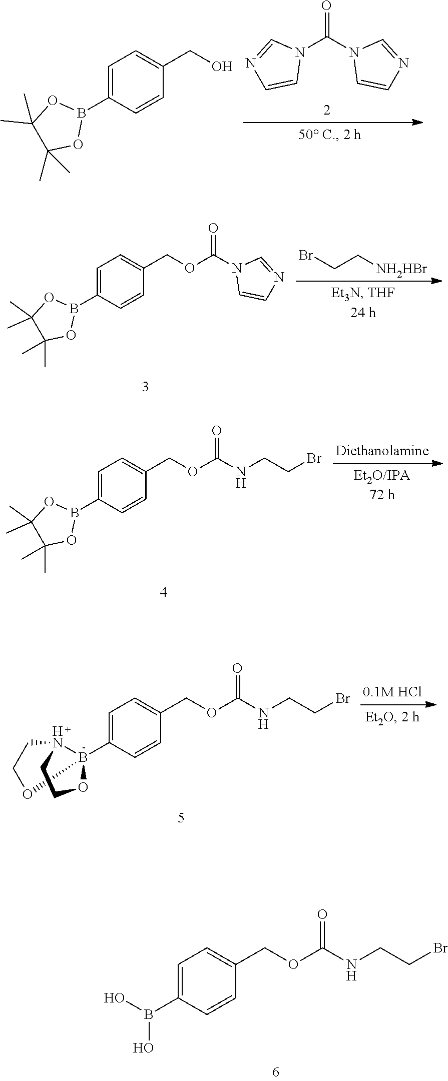

A 1 L three neck round bottom flask fitted with reflux condenser, nitrogen bubbler, and thermometer. Carbonyldiimidazole (18 g, 111 mmoles) in dry acetonitrile (200 mL) was added. Pinacol boronate phenyl alcohol (20 g, 85 mmol) was added to the reaction mixture under nitrogen atmosphere. The resulting mixture was heated to 50° C. until TLC analysis indicated no starting material, approximately 2 hours. After the completion of reaction, the reaction mixture was cooled to RT and concentrated under reduced pressure to afford a yellowish residue. The crude residue was triturated using pentane or hexane to afford 25 g of carbamate as a white solid.

A 500 mL 4 neck round bottom flask was flame dried and equipped with condenser, thermopocket, septum and magnetic stir bar. The system was evacuated with N₂ gas for 15 minutes. Boronate CDI (12 g, 36.5 mmol), 2-bromoethylamine hydrobromide (19 g, 92 mmol) and 120 mL (10.0 vol) dry DMF were charged to the reaction flask. The reaction mass was stirred for 10 minutes to yield a homogeneous clear solution. After that triethylamine (15 g, 146 mmol) was charged to the reaction flask by syringe. The heterogeneous mixture was stirred until completion of the reaction. After completion of the reaction as indicated by TLC, 360 mL diethyl ether charged to the reaction mass and stirred for 10 mins. The reaction mass was filtered and the filtrate was extracted with ice water, brine and dried over anhydrous sodium sulphate. The organic layer concentrated under reduced pressure to yield crude product, which was purified by silica gel chromatography to yield 5.3 g solid product.

A 1 L three neck round bottom flask was flame dried, fitted with nitrogen bubbler, thermometer and septum, and evacuated with nitrogen gas for 15 mins. To this flask was charged the bromo-pinacolyl boronate ester (5.3 g, 14 mmol), diethanol amine (1.6 g, 15 mmol), 10.6 ml IPA and 106 ml diethyl ether. under nitrogen atmosphere. The resulting mixture was stirred at room temperature for approximately 72 hours. After the completion of reaction, the reaction mixture was filtered, and product washed with diethyl ether. Finally, the product was dried under vacuum at 45° C. to yield 4 g white solid.

A 250 mL three neck round bottom flask was flame dried, fitted with nitrogen bubbler, thermometer and septum, and was evacuated with nitrogen gas for 15 minutes. To this flask was charged the bromo-pinacolyl boronate DEA complex (4.0 g, 11 mmol), 123 mL diethyl ether and 123 mL of 0.1 M aqueous HCl. The reaction was stirred at room temperature for 2 hours and kept overnight at room temperature. After the completion of reaction, 125 mL of ether was added and the layers were allowed to separate. The organic layer separated and aqueous layer was further extracted with ether. The combined organic layer was washed with brine and dried over Na₂SO₄. Finally, after concentrating organic layer, 2.7 g of the title compound was obtained as a white crystalline material.

### Example 5. Synthesis of 2-((4-(4,4,5,5-tetramethyl-1,3,2-dioxaborolan-2-yl)benzyl)oxy)ethan-1-amine

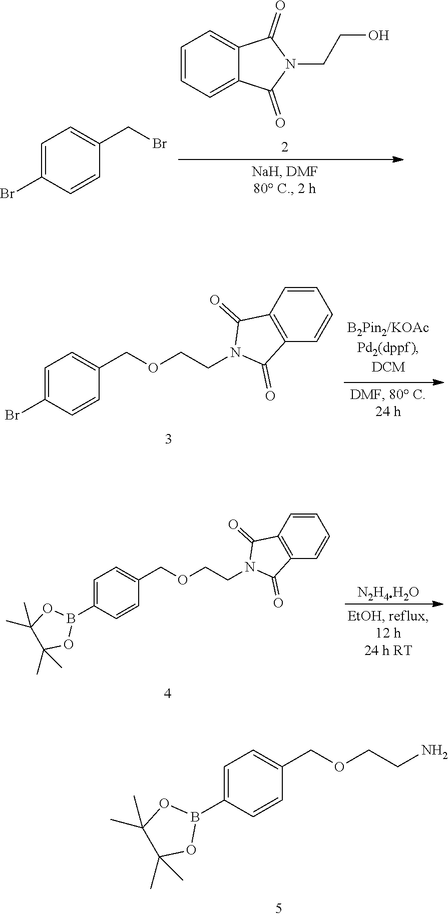

A 1 L three neck round bottom flask was flame dried and fitted with reflux condenser, nitrogen bubbler, thermometer and septum, and was evacuated with nitrogen gas for 15 mins. 125 mL dry DMF and NaH (6.24 g, 156 mmol) was added to the reaction flask, and the reaction flask was cooled to 0-5° C. N-(2-Hydroxyethyl)phthalimide (23 g, 120 mmol) dissolved in 68 ml DMF was slowly added to the reaction mixture and heated at 40° C. for 30 mins. 4-bromobenzyl bromide (30 g. 120 mmol) dissolved in 60 ml DMF was added slowly to the reaction mixture and heated to 70° C. for 2 hours. After the completion of reaction, the reaction mixture was cooled to RT and 500 mL water added. Product was extracted into ethyl acetate and ethyl acetate layer concentrated under reduced pressure to obtain pale yellow crude product. The crude residue was purified by silica gel column chromatography obtaining 13 g product.

A 1 L three neck round bottom flask was flame dried, fitted with reflux condenser, nitrogen bubbler, thermometer and septum, and was evacuated with nitrogen gas for 15 mins. 2-(2(4-bromobenzyl)oxyethylisoindoline-1,3-dione (13 g, 36 mmol), bis-pinacol boronate (10 g, 40 mmol), 10.6 g potassium acetate and PdCl₂(dppf) were charged to the reaction flask; DCM (1.47 g, 1.8 mmol) was added under nitrogen atmosphere. The resulting mixture was heated to 85° C. for 20 hours under continuous flow of nitrogen. After the completion of reaction, the flask was cooled to RT and 130 ml water was added. The product was extracted into ethyl acetate. The ethyl acetate layer was washed with water and brine and dried over sodium sulphate. The organic layer was concentrated under reduced pressure and the residue purified using silica gel column chromatography to obtain 12 g white solid product.

A 1 L four neck round bottom flask was fitted with reflux condenser, nitrogen bubbler, thermometer and septum, and 2-(2-(4,4,5,5-tetramethyl-1,3,2-dioxoboronate-2-yl)benzyl)oxy)ethyl)isoindoline-1,3-dione (6.5 g, 16 mmol) in 325 ml ethanol was added, then the reaction vessel was evacuated with nitrogen gas for 15 minutes. To this reaction solution was added 10 mL hydrazine hydrate at room temperature and reaction mixture was refluxed for 12 hours. The reaction mixture was cooled to room temperature and stirred at room temperature for 24 hours. After the completion of reaction, the reaction mixture was filtered through a celite pad and the mother liquor was concentrated under vacuum to obtain residue, which was dissolved in diethyl ether. The ether layer was washed with brine and contracted under vacuum to obtain 2.6 g of the title product as a white solid.

The examples herein are for illustrative purpose and are not meant to limit the scope of the invention as defined by the claims.

The invention claimed is:

1-7. (canceled)

8. A compound of Formula I 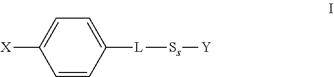
wherein
X is selected from a boronic acid group and a boronic acid pinacol ester group,
L is a linker,
S is a spacer, s is 0 to 10, and
Y is a chemically linkable end group.

9. (canceled)

10. The compound of claim 8 wherein L is selected from the group consisting of carbamate, ether, C₁-C₂₀ alkylenyl and —[—O—CH₂CH₂—]ₙ—, wherein n is 1-12.

11. The compound of claim 8 wherein S is selected from the group consisting of C₁-C₂₀ alkylenyl and —[—O—CH₂CH₂—]ₙ—, wherein
n is 1-12, and
s is 1-10.

12. The compound of claim 8 wherein Y is selected from the group consisting of halo, amino, carboxyl, C₂-C₁₀ alkynyl, hydroxyl, C₁-C₁₀ alkoxy, azido, sulfinate, thiol, fluorosulfate, and boronic acid.

13. The compound of claim 8 further comprising a molecule conjugated to the chemically linkable end group.

14. The compound of claim 13 wherein the molecule is selected from the group consisting of drugs, probes, dyes, peptides, proteins, drug candidates and natural products.

15. A compound of Formula II 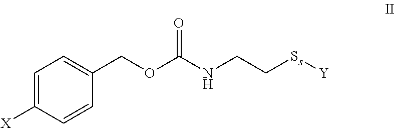
wherein
X is selected from a boronic acid group and a boronic acid pinacol ester group,
S is a spacer, s is 0 to 10, and
Y is a chemically linkable end group.

16. (canceled)

17. The compound of claim 15 wherein S is selected from the group consisting of C₁-C₂₀ alkylenyl and —[—O—CH₂CH₂—]ₙ—, wherein
n is 1-12 and
s is 1-10.

18. The compound of claim 15 wherein Y is selected from the group consisting of halo, amino, carboxyl, C₂-C₁₀ alkynyl, hydroxyl, C₁-C₁₀ alkoxy, azido, sulfinate, thiol, fluorosulfate, and boronic acid.

19. The compound of claim 15 further comprising a molecule conjugated to the chemically linkable end group.

20. The compound of claim 19 wherein the molecule is selected from the group consisting of drugs, probes, dyes, peptides, proteins, drug candidates and natural products.

21. A compound of Formula III 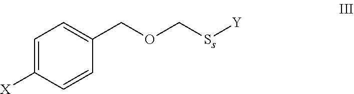
wherein
X is selected from a boronic acid group and a boronic acid pinacol ester group,
S is a spacer, s is 0 to 10, and
Y is a chemically linkable end group.

22. (canceled)

23. The compound of claim 21 wherein S is selected from the group consisting of C₁-C₂₀ alkylenyl and —[—O—CH₂CH₂—]ₙ—, wherein
n is 1-12, and
s is 1-10.

24. The compound of claim 21 wherein Y is selected from the group consisting of halo, amino, carboxyl, C₂-C₁₀ alkynyl, hydroxyl, C₁-C₁₀ alkoxy, azido, sulfinate, thiol, fluorosulfate, and boronic acid.

25. The compound of claim 21 further comprising a molecule conjugated to the chemically linkable end group.

26. The compound of claim 25 wherein the molecule is selected from the group consisting of drugs, probes, dyes, peptides, proteins, drug candidates and natural products.

27. A compound selected from the group consisting of (4-((((2-aminoethyl)carbamoyl)oxy)methyl)phenyl)boronic acid hydrochloride, (4-((((2-bromoethyl)carbamoyl)oxy)methyl)phenyl)boronic acid, 4-(4,4,5,5-tetramethyl-1,3,2-dioxaborolan-2-yl)benzyl (bromoethyl)carbamate, 4-(4,4,5,5-tetramethyl-1,3,2-dioxaborolan-2-yl)benzyl (2-aminoethyl)carbamate and 2-((4-(4,4,5,5-tetramethyl-1,3,2-dioxaborolan-2-yl)benzyl)oxy)ethan-1-amine.

28. A method of delivering a molecule to a nucleus in a cell, the method comprising contacting the cell with a compound of claim 13.

29-30. (canceled)

31. A method of delivering a molecule to a nucleus in a cell, the method comprising contacting the cell with a compound of claim 19.

32. A method of delivering a molecule to a nucleus in a cell, the method comprising contacting the cell with a compound of claim 25.
